(19) United States
(12) Patent Application Publication
(10) Pub. No.: US 2009/0016956 A1
(43) Pub. Date: Jan. 15, 2009

(54) Labeled Macrophages and Methods of Use Thereof

(76) Inventors: Robert Bender, Ottawa (CA); Allan M. Green, Cambridge, MA (US); Perry Kim, Inverary (CA); Robert Kisilevsky, Kingston (CA); Douglas S. McNair, Leawood, KS (US); Shui-pang Tam, Kingston (CA)

Correspondence Address: LAHIVE & COCKFIELD, LLP;FLOOR 30, SUITE 3000, ONE POST OFFICE SQUARE, BOSTON, MA, 02109, US

(21) Appl. No.: 12/110,865

(22) Filed: Apr. 28, 2008

Related U.S. Application Data
(60) Provisional application No. 60/926,489, filed on Apr. 27, 2007.

(51) Int. Cl. A61K 51/02; C12N 5/00; A61K 49/00; C12Q 1/02; A61K 35/12

(52) U.S. Cl. 424/1.17; 435/325; 424/9.2; 435/29; 424/93.7; 424/1.73

(57) ABSTRACT

Methods of making and using labeled macrophages are described.

### RELATED APPLICATIONS

This application claims priority to U.S. Provisional Patent Application No. 60/926,489; filed on Apr. 27, 2007. This application is related to U.S. patent application Ser. No. 11/582,857, filed on Oct. 17, 2006, which claims priority to U.S. Provisional Patent Application Ser. No. 60/836,520, filed on Aug. 8, 2006, U. S. Provisional Patent Application Ser. No. 60/728,027, filed on Oct. 17, 2005. The entire contents of each of these applications are hereby incorporated herein by reference.

### BACKGROUND OF THE INVENTION

Atherosclerosis is the process in which deposits of fatty substances, cholesterol, cellular waste products, calcium and other substances build up in the inner lining of an artery. The arterial buildup is called plaque and is usually found in large and medium-sized arteries. Atherosclerosis is a slow, progressive disease that may start in childhood and grow rapidly worse as with age.

Plaques can grow large enough to significantly reduce the blood's flow through an artery. However, most of the damage occurs when plaques become fragile and rupture. Plaques that rupture can cause blood clots to form that can block blood flow or break off and travel to another part of the body. When a blood clot blocks a blood vessel, serious health consequences can ensue. For example, if a clot or plaque blocks a blood vessel that feeds the heart or brain, a heart attack or stroke can occur. In addition, if blood supply to the arms or legs is reduced, difficulty walking and even gangrene can transpire.

Macrophages, which are cells within tissues that originate from monocytes, act in the body's defense mechanism by the phagocytosis of cellular debris and pathogens and the stimulation of lymphocytes and other immune cells. However, macrophages also are the predominant cells involved in creating the plaques that lead to atherosclerosis. Specifically, the initial damage to the blood vessel wall caused by the development of “fatty streak” (small subendothelial deposits of lipid) results in an inflammatory response. Monocytes enter the arterial wall from the blood stream, along with platelets that adhere to the site of the fatty streak. The monocytes thereafter differentiate into macrophages, which ingest oxidized low density lipoprotein (LDL), slowly turning into “foam cells.” Foam cells, which occur when macrophages arriving at sites of injury ingest cellular fragments for further processing and acquire a considerable cholesterol load, eventually die, and further propagate the inflammatory process. At the same time, smooth muscle proliferation occurs in response to cytokines secreted by damaged endothelial cells, which causes the formation of a fibrous capsule covering the fatty streak.

### SUMMARY OF THE INVENTION

Because diseases caused by atherosclerosis, such as coronary artery disease, are the leading cause of death in the United States, there is a need for a marker that allows for predictive assessment of the response of subjects to agents designed to reduce plaque by modification of the (reverse) cholesterol transport pathway (RCTP).

In one embodiment, the invention pertains to a method for labeling cells. The method includes contacting the cells with a cholesterol carrier that is internalized by the cells, such that the cells are labeled.

In another embodiment, the invention also pertains, at least in part, to a method for assessing the effectiveness of a test drug to modulate the cholesterol transport pathway in a subject. The method includes administering to the subject cells comprising labeled cholesterol; administering to the subject an amount of the test drug; and monitoring the time course of release of the labeled cholesterol in the subject.

In yet another embodiment, the invention also pertains, at least in part, to a diagnostic composition comprising cells, which comprise labeled cholesterol, and a pharmaceutically acceptable carrier.

In yet another embodiment, the invention also pertains, at least in part, to a composition comprising radiolabeled cholesterol and a substituted or unsubstituted cyclodextrin.

In another embodiment, the invention pertains, at least in part, to a method for labeling leukocytes in a biological sample by obtaining a biological sample from a subject; subjecting the sample to centrifugation to obtain a buffy coat; and contacting the leukocytes within the buffy coat with a cholesterol carrier that is internalized by the leukocytes.

In one embodiment, the invention pertains, at least in part, to a method for labeling leukocytes in a biological sample by obtaining a biological sample from a subject; subjecting the sample to centrifugation to obtain a buffy coat; removing the leukocytes from the buffy coat; and contacting the leukocytes with a cholesterol carrier that is internalized by the leukocytes.

In another embodiment, the invention pertains, at least in part, to a method for labeling monocytes by contacting the monocytes with a cholesterol carrier that is internalized by the monocytes.

In a further embodiment, the invention pertains, at least in part, to a method for labeling leukocytes by contacting the leukocytes with a cholesterol carrier that is internalized by the leukocytes.

In a one, the invention pertains, at least in part, to a method for labeling macrophages by contacting the macrophages with a cholesterol carrier that is internalized by the macrophages.

In yet another embodiment, the invention pertains, at least in part, to a kit comprising one or more labeled cholesterol compounds and one or more pharmaceutically acceptable cholesterol carriers, buffers, and/or media.

### BRIEF DESCRIPTION OF THE DRAWINGS

FIG. 1 is a graph illustrating the cholesterol efflux induced by P1 or P4 liposomes in CD1 mice loaded with ³H-cholesterol/methyl-cyclodextrin/J774 cells as a function of time.

FIG. 2 is a chart illustrating the cholesterol efflux induced by P1 or P4 liposomes in CD1 mice loaded with ³H-cholesterol/methyl-cyclodextrin/J774 cells as a function percent efflux of baseline.

FIG. 3 is a graph illustrating the time course of cholesterol efflux caused by saline (•), empty PC liposomes (∘) and liposomes containing PPL4 (▴) in rabbits injected with ³H cholesterol-labeled and loaded THP-1 cells at various time points.

FIG. 4 is a graph illustrating the time course of cholesterol efflux (after normalized against 24 hour baseline) caused by saline (•), empty PC liposomes (∘) and liposomes containing PPL4 (▴) in rabbits injected with ³H-cholesterol-labeled and loaded THP-1 cells at various time points.

FIG. 5 is a graph illustrating the time course of cholesterol efflux (dpm/μl plasma) caused by P1 and P4 peptide administration in animals injected with sonicated (∘) and intact living cells (∇). Control animals were treated with PBS after being injected with sonicated (•) and intact living cells (▾).

FIG. 6 is a chart illustrating the cholesterol efflux induced by PPL4, or mD27mer (a D-amino acid peptide that enhances CEH activity) in cells loaded with acetylated-LDL labeled with ³H-cholesterol. In vivo efflux was determined by measuring plasma ³H-cholesterol. Values were standardized to levels at 24 hours (i.e., percent efflux of baseline).

FIG. 7 is a graph illustrating the time course of cholesterol efflux (dpm/μl plasma) caused by native HDL (N-HDL) at doses of 200 (•) or 400 (∘) μg/mouse, or by acute-phase HDL (HDL-SAA) at doses of 200 (▾) or 400 (∇) μg/mouse. Controls were treated with saline (▪).

FIG. 8 is a graph illustrating the time course of cholesterol efflux (dpm/μl plasma) caused by liposome formulated (∘) and non-liposome formulated (•) Sandoz 58-035, a small molecule ACAT inhibitor. Controls were treated with saline (▾).

FIGS. 9 and 10 are charts illustrating the in vivo tissue distribution of macrophages over a 24 hr period. Cells were prelabeled with ³H cholesteryl ether.

### DETAILED DESCRIPTION OF THE INVENTION

There is a general need for a marker which will allow predictive assessment of the response of subjects to agents designed to reduce plaque by modification of the (reverse) cholesterol transport pathway (RCTP). Advantageously, the marker of the invention crosses species boundaries (e.g., between humans, monkeys, cats, and other mammals etc.) and is quantitatively predictive.

The term “plaque” includes, for example, an atherosclerotic plaque.

In one embodiment, the invention pertains to a ‘modified in vivo assay’ (MIVA). MIVA may be used to assess or study compounds and/or compound formulations in development. One feature of MIVA is its ability to predict a compound's efficacy in modifying (e.g., reducing, preventing, inhibiting, or regressing) atherosclerosis.

Another salient feature of MIVA is its ability to predict a compound's efficacy for the treatment of macrophage-related diseases.

The term “macrophage-related diseases” include diseases associated with macrophages such as atherosclerosis and other disease associated with abnormal cholesterol metabolism. Examples of macrophage-related diseases, include, but are not limited to, heart disease, peripheral artery disease, and stroke (e.g., ischemic stroke, hemorrhagic stroke).

MIVA results have been shown to correlate with long term in vivo anti-atherosclerosis studies in mice (see, for example, U.S. Pat. No. 7,291,590; U.S. application Ser. No. 11/268,690; and U.S. application Ser. No. 11/872,309, the contents of each of which are hereby incorporated by reference in their entirety).

In one embodiment, the invention pertains to a method for labeling cells by contacting the cells with a cholesterol carrier that is internalized by the cells, such that the cells are labeled.

The term “cells” includes cells from humans and other subjects, including, but not limited to cats, dogs, ferrets, farm animals (cows, sheep, pigs, horses, goats, etc.), lab animals (rats, mice, monkeys, etc.), and primates (chimpanzees, humans, gorillas). Examples of suitable cells types include, for example, leukocytes (i.e., white blood cells), macrophages (e.g., autologous, peripheral or peritoneal macrophages), monocytes, lymphocytes, neutrophils, eosinophils, and/or basophils.

In another embodiment, the leukocytes are in the buffy coat of anti-coagulated blood.

The term “buffy coat” includes the fraction of an anticoagulated blood sample after centrifugation that comprises leukocytes (e.g., white blood cells) and platelets.

In another embodiment, the invention includes a method of loading of cells, (e.g., macrophages in culture) with labeled cholesterol, e.g., radiolabeled cholesterol, by exposing the cells to autologous red cell membrane fragments which have been equilibrated with labeled cholesterol. The exposed cells phagocytose the red cell membrane fragments and internalize and store the labeled cholesterol becoming, in effect, foam cells. These ‘loaded’ cells are then administered, e.g., intraveneously injected, into a subject and allowed to distribute throughout the subject and settle within its organs (e.g., heart, lung, kidney, liver). A base level of cholesterol release is observed. After an appropriate length of time, the subject is treated with the test agent, or compound of interest and the release of label (e.g., labeled cholesterol) is measured and/or monitored as a function of time, dose, or dosing regimen. The appropriate length of time may be approximately twenty four hours, although it may also be longer or shorter, if so desired.

The term “settle” includes the movement of cells out of the vasculature (e.g., out of the lumen of the arterial or venous system) and entering and residing in the extravascular areas of the respective organs.

Stimulation of cholesterol release from the introduced loaded cells results in a transient increase in the amount of circulating label over baseline. Measurement of the kinetics of label release is carried out by direct analysis of samples of circulating whole blood, plasma, serum, feces, urine, saliva and/or cerebral spinal fluid.

The methods of the invention may be used for investigating agents which act directly on steps or processes along the reverse cholesterol transport pathway (RCTP). Molecules or cells that play an important role in the RCTP are for example, high density lipoprotein (HDL), low density lipoprotein (LDL), endothelial cells, smooth muscle cells, hepatocytes, intestinal cells and macrophages.

In one embodiment, the invention pertains to a method for assessing the effectiveness of a test drug to modulate the cholesterol transport pathway in a subject comprising the steps of administering to the subject cells (e.g., leukocytes, macrophages or monocytes) comprising labeled cholesterol; administering to the subject an amount of a test drug; and monitoring the time course of release of the labeled cholesterol in the subject, thus assessing the effectiveness of a test drug to modulate the cholesterol transport pathway in a subject. Appropriate methods of administrating cells to the subject include intravenous administration.

This method may also further include the step of measuring the time course of release of the labeled cholesterol prior to the administration of the test drug and/or administering additional therapeutic or diagnostic agents in combination with the cell and/or the test drug. The additional therapeutic or diagnostic agent may be administered to the subject orally or intravenously. In one embodiment, the cells are labeled by methods described herein. In a further embodiment, macrophages are from a different species than said subject, provided that when the subject is human the macrophages are not from a different species.

Molecules or compounds that have shown to be effective in modulation of the RCTP include ACAT inhibitors (e.g., P1, Sandoz 58-035), CEH enhancers (e.g., P4, PPL4), high density lipoprotein (HDL) and agents that enhance the ABCA1 transporter (e.g., PPL4).

The methods of the invention may also be used to probe events further downstream insofar as they result in changes in the fluxes of cholesterol to or from the macrophage (plaque) reservoirs.

Examples of agents which may affect fluxes of cholesterol include, but are not limited to, compounds that inhibit cholesterol ester transfer protein (CETP), compounds that prevent cholesterol absorption (e.g., ezetimibe), and bile acid resin and other types of cholesterol sequestrants (e.g., cholestimide, cholestyramine).

By combining MIVA with pulse-label methods in which labeled cholesterol is introduced orally or at other points in the cholesterol transport pathway, it is possible to improve the ability to resolve the behavior of multiple interacting reservoir components.

As a number of agents targeted to the RCTP are under development, assessment of the effect of these on plaque in patients, or patient clinical outcome (e.g., morbidity, mortality) is of increasing interest. The definitive measurement at autopsy is, understandably, not a desired data set in a clinical setting. Certain imaging techniques (e.g., multi-detector CT, MRI and PET) may fall short of the sensitivity and resolution needed for dynamic assessment although recent studies provide evidence that they are getting closer to clinical utility for diagnosis; intravascular ultrasound (IVUS) measurements are difficult and invasive.

MIVA has the potential to provide a clinical tool for studies of agents in development as well as a method to predict individual response to specific agents or combination therapies. This type of data may be used by companies to stratify subjects and may be used as an additional inclusion/exclusion criteria for entry into clinical trials for drug safety or efficacy assessment.

The invention also pertains to computational methods for assessing the nature of the response of macrophages based on the kinetic data on label concentration in the blood of a subject.

The term “cholesterol carrier,” refers to any medium which is capable of carrying labeled cholesterol such that the labeled cholesterol is brought into contact with a macrophage and is internalized. Examples of cholesterol carriers include cell membrane portions, e.g., autologous red blood cell fragments equilibrated with labeled cholesterol, acetylated LDL equilibrated with labeled cholesterol, unilamellar or multilamellar liposomes containing labeled cholesterol, or substituted or unsubstituted cyclodextrins, e.g., substituted or unsubstituted α-cyclodextrin, β-cyclodextrin or γ-cyclodextrin. In one embodiment, the cyclodextrin is β-methyl-cyclodextrin.

In another embodiment, the invention pertains, at least in part, to a method for labeling leukocytes in a biological sample by obtaining a biological sample from a subject; subjecting the sample to centrifugation to obtain a buffy coat; and contacting the leukocytes within the buffy coat with a cholesterol carrier that is internalized by the leukocytes.

In one embodiment, the invention pertains, at least in part, to a method for labeling leukocytes in a biological sample by obtaining a biological sample from a subject; subjecting the sample to centrifugation to obtain a buffy coat; removing the leukocytes from the buffy coat; and contacting the leukocytes with a cholesterol carrier that is internalized by the leukocytes.

In another embodiment, the invention pertains, at least in part, to a method for labeling monocytes by contacting the monocytes with a cholesterol carrier that is internalized by the monocytes.

The term “biological sample” includes, but is not limited to, blood, urine, feces, spinal fluid and saliva.

The term “labeled cholesterol,” includes a cholesterol compound that has been modified to include a means of detecting the cholesterol compound. For example, the cholesterol compound may include a fluorescent label, e.g., NBD, a spin probe or may be labeled with a stable or radioactive isotope label, e.g., tritium, deuterium or ¹⁴C. In a further embodiment, the labeled cholesterol is ³H-cholesterol or ¹⁴C-cholesterol. Advantageously, the radiolabel is selected such that it has a long half life and can be used in substantially non-toxic amounts. In addition, the term “labeled cholesterol” may also include “cholesterol-like” compounds that behave very similarly to cholesterol within the cell or subject. Cholesterol-like compounds may be used in replace of, or in conjunction with, cholesterol and would be metabolized or processed in a similar manner to cholesterol. Examples of cholesterol-like compounds include, for example, cholesterol esters or cholesterol ethers. Cholesterol-like compounds may be modified chemically or enzymatically and detected in a similar manner to cholesterol in the MIVA, as described above.

The substantially non-toxic, low or ultra-low amount/concentration of labeled cholesterol may be significantly below that previously used in animal studies. A non-toxic, low or ultra-low amount/concentration of radiolabel, and the resulting radiation exposure, that one may use in human MIVA experiments may be as low as, or lower than, normal human daily exposure. A normal human daily exposure of ionizing radiation may include, but is not limited, to cosmic rays during a commercial plane flight (e.g., five to ten microsieverts), or a chest x-ray (e.g., 50 microsieverts.). Non-toxic amounts of ¹⁴C or tritium labeled cholesterol may be useful when conducting MIVA to measure assessment of the response of experimental subjects/patients to agents designed to reduce plaque by modification of the (reverse) cholesterol transport pathway (RCTP). Assessment can include the collection of blood/plasma/serum, urine, feces, saliva, or cerebral spinal fluid from individuals during MIVA.

The term “internalized” includes any method by which cells take up the cholesterol carrier containing the labeled cholesterol, e.g., phagocytosis, receptor-mediated intracellular internalization and protein transporter-mediated intracellular internalization. Upon internalizing the cholesterol carrier comprising the labeled cholesterol, the cells are then labeled.

In another embodiment, the invention pertains, at least in part, to a method for assessing the effectiveness of a compound or test drug, or combination of compounds to modulate the RCTP in a subject, comprising the steps of administering to said subject a preparation of autologous macrophages comprising labeled cholesterol; administering to said subject an amount of said test drug; and monitoring the time course of release of said labeled cholesterol in said subject, thus assessing the effectiveness of a test drug to modulate the RCTP in a subject. In a further embodiment, the method for assessing the effectiveness of a test drug to modulate the cholesterol transport pathway in a subject may further comprise the step of measuring the time course of release of said labeled cholesterol prior to the administration of said test drug. In one embodiment, the macrophages are labeled by contacting the macrophages with a cholesterol carrier that is internalized by the macrophages, such that the macrophages are labeled.

In another embodiment, the invention pertains, at least in part, to a method for assessing the effectiveness of a compound or test drug, or combination of compounds to modulate the cholesterol efflux capability of human cells (e.g., monocytes or macrophages), comprising the steps of administering to said non-human animal a preparation of human monocytes or macrophages comprising labeled cholesterol; administering to said non-human animal an amount of said test drug; and monitoring the time course of release of said labeled cholesterol in said non-human animal, thus assessing the effectiveness of a test drug to modulate the in vivo cholesterol efflux capability of said human monocytes or macrophage, and also to generally assess the effect on the RCTP. In a further embodiment, the method for assessing the effectiveness of a test drug to modulate the in vivo cholesterol efflux capability of said human monocytes or macrophages, in a non-human animal may further comprise the step of measuring the time course of release of said labeled cholesterol prior to the administration of said test drug. In one embodiment, the monocytes or macrophages are labeled by contacting the cells with a cholesterol carrier that is internalized by the cells, such that the cells are labeled.

In another embodiment, the invention pertains, at least in part, to a kit for conducting the MIVA. The components of the kit may include, but are not limited to, buffer or media that maintain the integrity and vitality of the cells such as macrophages or monocytes (e.g., phosphate buffered saline (PBS), HEPES solution, Hank's balanced salts, Dulbeco minimal essential medium (DMEM), minimum essential medium (MEM) solution), labeled cholesterol, a second or possibly third labeled cholesterol, and/or one or more cholesterol carriers. The cholesterol carrier(s) within the kit may, or may not already be carrying the labeled cholesterol.

The term “monitoring” includes any analytical methods known in the art for detecting radiolabeled compounds in samples. Advantageously, the radiolabel can be detected by analytical methods. Examples of analytical methods which can be used to monitor the labeled cholesterol and macrophages include but are not limited to mass spectroscopy, e.g., accelerator mass spectrometry (AMS).

Examples of subjects include mammals (e.g., cats, dogs, ferrets, etc.), farm animals (cows, sheep, pigs, horses, goats, etc.), lab animals (rats, mice, monkeys, etc.), and primates (chimpanzees, humans, gorillas). In one embodiment, the subject is a human. The subject may have an atherosclerotic condition or be at risk of suffering from an atherosclerotic condition. A subject at risk of suffering from an atherosclerotic condition may or may not show symptoms of the atherosclerotic condition. In certain embodiments, the term may also include transgenic laboratory animals, such as mice, rats, rabbits, etc.

In another embodiment, the macrophages are human macrophages. Macrophages from one species may be administered to another species in order to measure the effectiveness of a test drug to modulate the cholesterol transport pathway in a subject prior to an immune response. For example, human macrophages may be administered to a mammal other than a human, although macrophages from a mammal other than a human are not administered to a human.

The cells may be administered to the subject by any appropriate method known in the art. For example, the macrophages may be injected or administered intravenously, arterially or peritoneally.

In another embodiment, the method for assessing the effectiveness of a test drug to modulate the RCTP in a subject may further comprise administering additional therapeutic or diagnostic agents in combination with the macrophages or the test drug. The additional therapeutic or diagnostic agent may be administered intravenously or by any other technique applicable.

In one embodiment, the invention pertains, at least in part, to a diagnostic composition comprising a pharmaceutically acceptable carrier and cells comprising labeled cholesterol. In another embodiment, the pharmaceutically acceptable carrier is acceptable for intravenous administration. In a further embodiment, the labeled cholesterol is labeled with a stable isotope or a radiolabel, e.g., deuterium, tritium or ¹⁴C.

The language “pharmaceutically acceptable carrier” includes substances capable of being coadministered with the cells and/or cholesterol carriers of the invention, and which allow both to perform their intended function, e.g., label cells. Suitable pharmaceutically acceptable carriers include but are not limited to water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with the active compounds of the invention.

MIVA may also be used to assess the suitability of a particular therapeutic intervention for a particular subject. In one embodiment, the method pertains to a method for assessing the suitability of a particular therapy by administering to a subject a labeled macrophage in combination with a therapeutic intervention, and monitoring the time course of release of labeled cholesterol from said labeled macrophages to determine the suitability of a particular therapy for a particular subject.

### EXEMPLIFICATION OF THE INVENTION

### EXAMPLE 1

### Cholesterol Efflux Induced in Mice

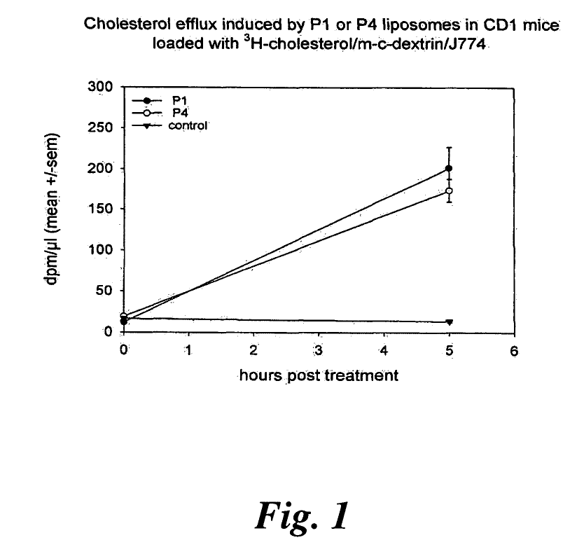

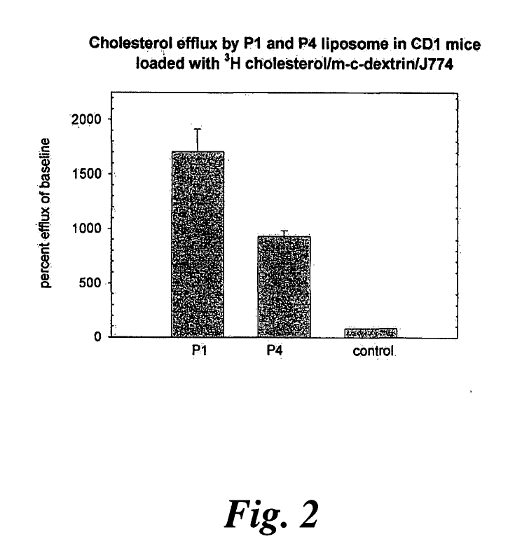

In order to demonstrate that different cholesterol loading techniques are equally effective in the MIVA, a sample of J774 cells were pre-loaded and labeled with a methylcyclodextrin-[³H]-cholesterol complex. The cells labeled with the cholesterol complex were then administered to CD1-mice intravenously. A modified in vivo assay was carried out using liposomal formulations containing either P1 (the mouse form of the acyl CoA:cholesterol acyl transferase [ACAT] inhibitor peptide) or P4 (the mouse form of the cholesterol ester hydrolase [CEH] enhancer peptide) with measurements of dpm/μl plasma taken five hours after treatment. The results of this assay can be seen in FIG. 1 and FIG. 2.

### EXAMPLE 2

### Cholesterol Efflux Induced in Rabbits

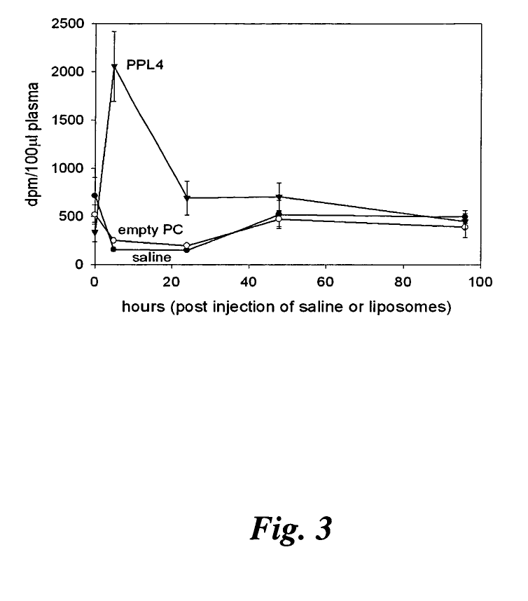

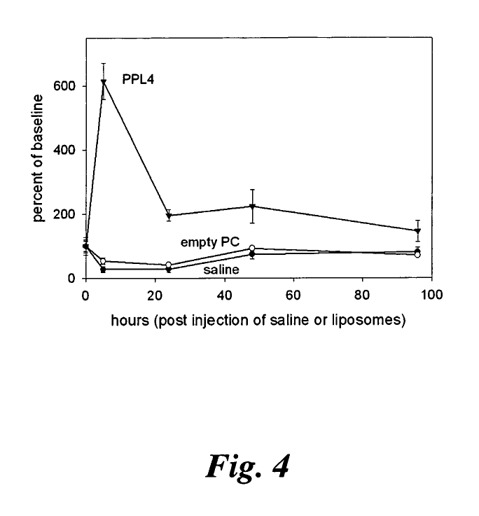

In order to demonstrate that cells of various animal species may be used in the MIVA, samples of THP-1 cells were differentiated into macrophages by the treatment with PMA. The macrophages were then loaded with ³H-cholesterol-methyl-cyclodextrin (0.1 mM) prior to injection of rabbits. Upon injection into rabbits, the time course of cholesterol efflux caused by a saline control, empty PC liposomes, and liposomes containing PPL4 (the human form of the cholesterol ester hydrolase [CEH] enhancer peptide) was monitored over a period of 100 hours. The results of this assay can be seen in FIG. 3 and FIG. 4. These results show that cells from one animal species can be used in a different animal species. As shown in this example, human macophages were used rabbits, in contrast with Example 1, in which mouse macrophages were used in the same species (e.g., mice).

### EXAMPLE 3

### Living vs Dead Cells in the MIVA

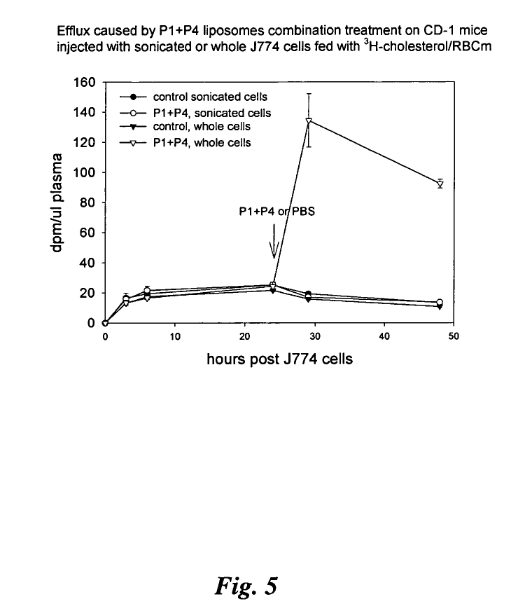

The following example demonstrates that cholesterol efflux is a function of living cells that have been injected into the animal. J774 cells were maintained in 6-well plates and the cells were cholesterol loaded with red blood cell (RBC) membrane fragments (175 μg) equilibrated with labeled [³H]-cholesterol (1.0 μCi/well). The cells were then washed extensively with phosphate-buffered saline (PBS) to remove the unincorporated label. Cells were then scraped off each well into 0.1 ml PBS. Half of the cells were disrupted by sonication (10×1 sec. intervals) prior to injection into the mice. The other half of the labeled intact living cells (one million cells/mouse) were injected intravenously into the animals. After injection of either sonicated or intact living cells, the animals were then administered liposomes containing P1 and P4. In vivo cholesterol efflux was then determined by sampling blood samples as indicated in FIG. 5 over a 48 hour period. Plasma dpm/μl plasma was determined using liquid scintillation. These results illustrate that cholesterol efflux is not due to phagocytosis of sonicated cell debris by resident endogenous macrophages followed by release of labeled cholesterol.

### EXAMPLE 4

### Macrophage Loading with Acetylated LDL

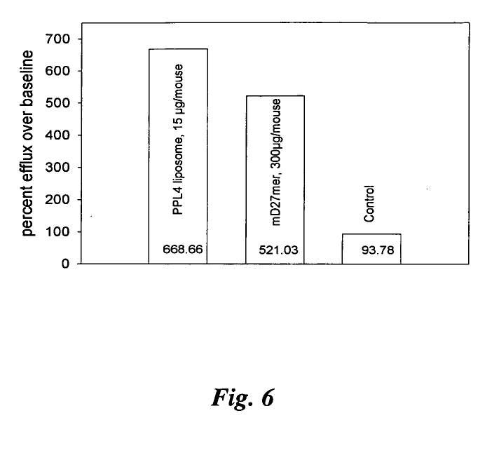

An additional technique for loading cholesterol in the MIVA was illustrated in the following example. J774 macrophages were treated similarly to that described in Example 3. However in this experiment macrophages were loaded acetylated-LDL labeled with [³H]-cholesterol (100 μg). After 48 hours, the cells were washed extensively with PBS to remove the unincorporated label. One million cells in 0.2 ml of PBS were injected into each animal (total 12) through the tail vein. After 24 hours post-injection of cells, the animals were divided into 3 groups of 4. The control group received peptide-free liposomes (100 μl per mouse). The other two groups received either liposomes containing PPL4 (15 μg/mouse) or mouse D27-mer peptide (300 μg/mouse, a CEH enhancer peptide). In vivo cholesterol efflux was then determined as described in Example 3 and the results of this example are shown in FIG. 6.

### EXAMPLE 5

### HDL Efficacy in the MIVA

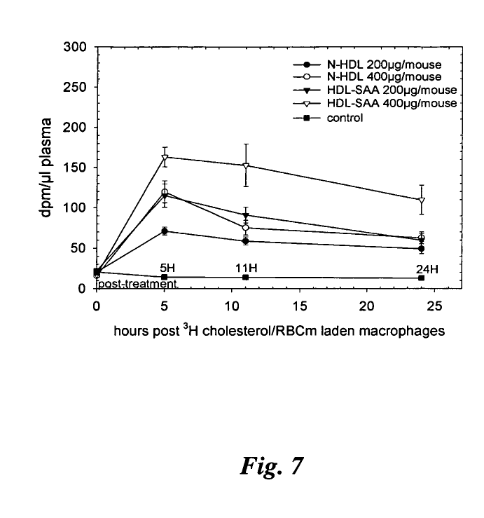

In order to demonstrate that native HDL and acute phase HDL are effective in the MIVA, native HDL (N-HDL) and acute-phase HDL (HDL-SAA) were isolated from normal and inflamed mice as described previously (Tam et al. J. Lipid Res. 2002, 43:1410-1420). To determine cholesterol export in vivo, J774 macrophages were cholesterol loaded with RBC membranes and [³H]-cholesterol as described previously. One million labeled cells in 0.2 ml PBS were injected into each mouse via the tail vein. After 24 hours, five groups of four animals were administered intravenously, via the tail vein with 100 μl PBS per mouse (control), 200 μg N-HDL in 0.1 ml PBS, 400 μg N-HDL in 0.1 ml PBS, 200 μg HDL-SAA in 0.1 ml PBS or 400 μg HDL-SAA in 0.1 ml PBS. At various time points, approximately 25 μl of blood were collected from the tail vein of each animal into heparinized capillary tubes and then centrifuged for 5 min to separate red blood cells from plasma. Cholesterol efflux was determined by liquid scintillation counting and the results are shown in FIG. 7. This data supports the use of the MIVA for investigating the potential efficacy of different agents that work on various steps, or pathways in the RCTP.

### EXAMPLE 6

### Small Molecule ACAT Inhibitor in the MIVA

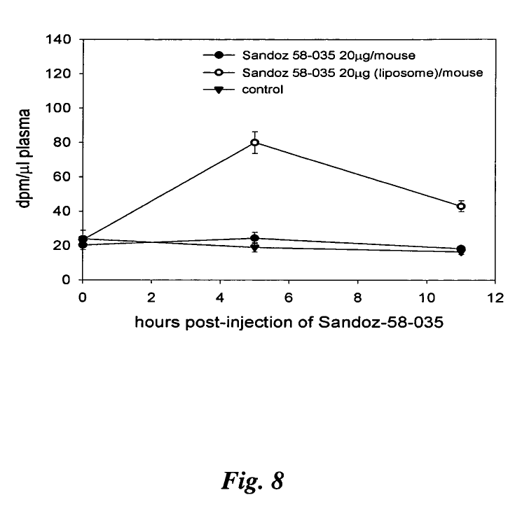

The effectiveness of small molecule ACAT inhibitors and CEH enhancer (e.g. P4, PPL4) peptide molecules in the MIVA were investigated as follows. The small molecule ACAT inhibitor (Sandoz 58-035) was first dissolved in dimethyl sulphoxide at a concentration of 2 mg/ml. For non-liposome formulated 58-035, 10 μl of the stock solution (2 mg/ml) was diluted with 190 μl PBS to give a solution of 20 μg/200 μl. Thus, in this group of animals, 200 μl of solution containing 20 μg of 58-035 was injected into each mouse through the tail vein. Liposomes were prepared as follows: Phospholipid (33.9 mg) and cholesterol (4.83 mg) were dissolved in choloroform and dried with nitrogen. For 10 ml liposomes, the thin film of dried lipid was hydrated with PBS containing 58-035 and cholic acid (53.75 mg). To make this solution, 0.5 ml of the 58-035 stock solution (2 mg/ml) in DMSO was diluted with 9.5 ml PBS containing 53.75 mg cholic acid. To form the liposomes, the dried lipids were incubated with PBS/cholic acid solution containing 1 mg 58-035 overnight at 4° C. by vortexing. The liposomes were then dialyzed extensively with 4 changes of 1 L PBS to remove cholic acid and unbound 58-035. The concentration indicated in FIG. 8 represent 100% incorporation of 58-035. This data supports the use of the MIVA for investigating the potential efficacy of different agents that work on various steps, or pathways in the RCTP.

### EXAMPLE 7

### Cell Kinetics

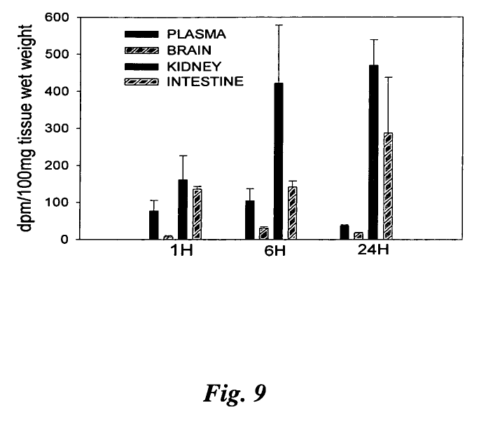

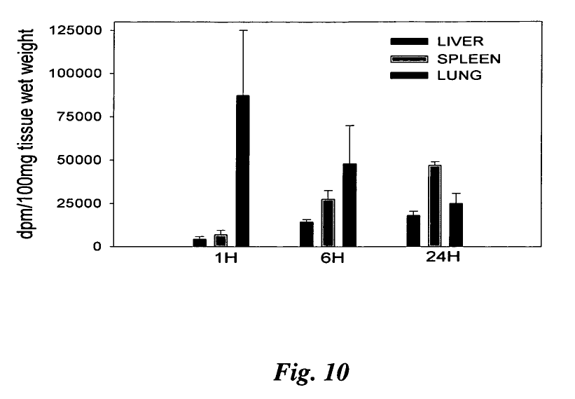

In order to demonstrate that the radioactivity detected within the organs of a subject is a direct measure of the amount of loaded cells residing in the extravascular regions of the various organs, J774 cells were labeled with [³H]-cholesteryl ether (0.5 μCi/ml) overnight. The cells were then washed with PBS extensively to remove the unincorporated label. One million of the labeled cells in 0.2 ml of PBS were then injected intravenously into each mouse through the tail vein. After 24 hours, post-injection of the cells, animals were perfused with PBS to remove the blood and then various organs were extracted from the animals and weighed. A portion of each organ was solubilized and the radioactivity of the samples were then determined by scintillation counting. Radioactivity is expressed as dpm/100 mg protein or for plasma 100 μl of plasma. The results of this example are shown in FIGS. 9 and 10. This data supports the use of the MIVA to measure the ability of agents to induce extravascular cholesterol mobilization and thus determine the potential efficacy of agents that work on various steps, or pathways in the RCTP.

### EQUIVALENTS

Those skilled in the art will recognize, or be able to ascertain using no more than routine experimentation, many equivalents to the specific embodiments of the invention described herein. Such equivalents are intended to be encompassed by the following claims. The entire contents of all references, patents and published patent applications cited throughout this application are hereby incorporated by reference.

1. A method for labeling cells, comprising contacting said cells with a cholesterol carrier that is internalized by the cells, such that said cells are labeled.

2. The method of claim 1, wherein said cholesterol carrier is a cell membrane portion equilibrated with labeled cholesterol.

3. The method of claim 2, wherein said cell membrane portions comprise autologous red blood cell fragments.

4. The method of claim 1, wherein said cholesterol carrier is selected from the group consisting of acetylated LDL equilibrated with labeled cholesterol, unilamellar or multilamellar liposomes containing labeled cholesterol, or a substituted or unsubstituted α-cyclodextrin, β-cyclodextrin and γ-cyclodextrin.

5. The method of claim 4, wherein said cyclodextrin is β-methyl-cyclodextrin.

6. The method of claim 1, wherein said labeled cholesterol is labeled with a stable or radioactive isotope label.

7. The method of claim 6, wherein said radiolabel is ¹⁴C or ³H.

8. A method for assessing the effectiveness of a test drug to modulate the cholesterol transport pathway in a subject, comprising:
administering to said subject cells comprising labeled cholesterol;
administering to said subject an amount of said test drug; and
monitoring the time course of release of said labeled cholesterol in said subject, thus assessing the effectiveness of a test drug to modulate the cholesterol transport pathway in a subject.

9. The method of claim 8, further comprising a step of measuring the time course of release of said labeled cholesterol prior to the administration of said test drug.

10. The method of claim 8, further comprising administering additional therapeutic or diagnostic agents in combination with the cell and/or the test drug.

11. (canceled)

12. The method of claim 8, wherein the time course of release of said cholesterol is monitored using mass spectroscopy.

13. (canceled)

14. (canceled)

15. The method of claim 8, wherein said cells are labeled by the method of claim 1.

16. (canceled)

17. The method of claim 1, wherein said cells are leukocytes in a buffy coat, monocytes or macrophages.

18.-23. (canceled)

24. A method for labeling leukocytes in a biological sample, comprising:
obtaining a biological sample from a subject;
subjecting said sample to centrifugation to obtain a buffy coat; and
contacting said leukocytes within said buffy coat with a cholesterol carrier that is internalized by said leukocytes, such that said leukocytes are labeled.

25.-30. (canceled)

31. A diagnostic composition comprising a pharmaceutically acceptable carrier and cells comprising labeled cholesterol for administration to a subject.

32. (canceled)

33. The diagnostic composition of claim 31, wherein said labeled cholesterol is labeled with a stable isotope or a radioactive isotope label.

34. The diagnostic composition of claim 31, wherein said labeled cholesterol is labeled with ¹⁴C or ³H.

35. (canceled)

36. A composition comprising radioactive isotope labeled cholesterol and a substituted or unsubstituted cyclodextrin.

37. The composition of claim 36, wherein said composition comprises ³H-cholesterol/methyl-cyclodextrin or ¹⁴C-cholesterol/methyl-cyclodextrin.

38.-40. (canceled)

41. A kit comprising one or more labeled cholesterol compounds and one or more pharmaceutically acceptable cholesterol carriers, buffers, and/or media.
